(19) United States
(12) Patent Application Publication
(10) Pub. No.: US 2008/0188416 A1
(43) Pub. Date: Aug. 7, 2008

(54) TISSUE FILLERS AND METHODS OF USING THE SAME

(75) Inventor: Eric F. Bernstein, Gladwyne, PA (US)

Correspondence Address: FISH & RICHARDSON PC, P.O. BOX 1022, MINNEAPOLIS, MN, 55440-1022, US

(73) Assignee: Freedom-2, Inc., Cherry Hill, NJ (US)

(21) Appl. No.: 12/026,457

(22) Filed: Feb. 5, 2008

Related U.S. Application Data
(60) Provisional application No. 60/888,260, filed on Feb. 5, 2007.

(51) Int. Cl. A61K 38/30; A61K 9/00; A61K 47/42; A01N 1/02; A61K 47/36; A61K 47/12

(52) U.S. Cl. 514/12; 424/484; 424/486; 424/487; 514/773; 514/777; 514/784; 435/1.1

(57) ABSTRACT

The invention relates to tissue filler compositions that are alterable or removable on demand. Also provided are methods for filling or augmenting tissue using the tissue fillers described herein, and correcting such tissue implants as required.

### CLAIM OF PRIORITY

This application claims the benefit of U.S. Provisional Patent Application Ser. No. 60/888,260, filed on Feb. 5, 2007, the entire contents of which are hereby incorporated by reference.

### FIELD OF THE INVENTION

This invention relates to tissue fillers for biomedical and other applications in humans and animals. The present disclosure also relates to methods of augmenting tissue in humans, such as the skin, subcutaneous fat, gums, urinary bladder and the like, by implanting or injecting such fillers into the tissue.

### BACKGROUND

In general, one objective of inserting or implanting a filler agent into a tissue, e.g., an organ, including the skin, is either to fill an area in which there is currently a deficit of material that should normally be present, or to produce a desired structural change in an organ system. However, one problem that plagues the use of fillers in humans and other mammals is the digestion and/or removal by other means of this dermal filler by the body. For instance, the human body often reacts to foreign substances by producing an inflammatory response, and removing or digesting the added material.

There are various known filler materials, such as collagen, elastin, fibrillin, fibulin, decorin, biglycan, hyaluronic acid, calcium hydroxyapatite, silicone, cells, and poly L-lactic acid, which have been used to augment the skin and treat various cosmetic conditions. When injected into the skin, however, these materials are often not removable or correctable at will in case the implantation procedure is not carried out as intended. This can result in unwanted bumps or other types of protrusions, which are difficult, if not impossible, to remedy without surgical intervention.

Also, conventional tissue fillers, such as dermal fillers, require repeated applications to maintain the desired appearance of the skin as the filler material degrades, is absorbed, or is otherwise dispersed in the body. Thus, repeated treatments, which are expensive and may cause a substantial amount of discomfort, are often needed.

### SUMMARY

In general, the present invention provides tissue filler compositions that include matrix materials, e.g., in the form of microparticles, that encapsulate, incorporate, entrap, or complex with, one or more tissue filler materials, to provide tissue filler compositions that have a longer period of efficacy than those presently known and that can be removed and/or altered after implantation into the tissue, e.g., the skin, of a patient. The tissue filler compositions can include such matrix-associated tissue filler materials alone or in combination with any one or more additional tissue filler materials, which can be the same or different from the tissue filler materials associated with the matrix materials.

After the tissue filler compositions are implanted, they can be altered by the application of a specific energy, e.g., a laser beam of a specific wavelength, to disrupt and remove the matrix materials, e.g., microparticles. When the microparticles are treated, not only are they disrupted to be digested or physically removed from their present location within the tissue by natural processes within the body, but some of the non-microparticle-associated tissue filler material will also be digested away, because disrupting the microparticles results in an inflammatory response that causes the body to remove at least some of the non-microparticle-associated tissue filler materials. Various types of tissue filler materials can be used alone or in combination with such a matrix to form microparticles.

In one aspect, the invention features tissue filler compositions including a first tissue filler material, and a matrix-associated tissue filler material including a matrix material and a second tissue filler material, wherein the matrix-associated tissue filler is indispersible when implanted into living tissue, but becomes dispersible when the matrix material is disrupted or when the second tissue filler material is separated from the matrix material.

In certain embodiments, the tissue filler compositions have matrix-associated tissue filler materials that further include a discrete absorption component. In various embodiments, the first and second tissue filler materials can be made of the same or different materials. In some embodiments, the first tissue filler material and the matrix-associated tissue filler material are mixed in a ratio of about 3:1 to about 1:3, e.g., about 1:1.

The matrix materials used in these compositions can include one or more of a polymethyl methacrylate, polyethylene vinyl-acetate, polytetrafluoroethylene, polyvinylchloride, polyamide, polyethylene, polysulfone, polyethersulphone, polypropylene, polydimethylsiloxane, polystyrene, polyphenylene oxide, cellulose, polypropylene oxide, polyvinylidene fluoride, and a polybutylenepolymethyl methacrylate, or mixtures thereof. In certain embodiments, the matrix materials can include (in addition or alternatively) one or more of an alginate, poly(vinyl alcohol), polyanhydride, polylactide, poly(glycolic acid), poly-L-lactic acid, poly D-lactic acid, poly-lactic-glycolic acid, and a triblock copolymer of poly caprolactone-polyethylene glycol-poly caprolactone, or mixtures thereof.

In these tissue filler compositions, either one or both of the first and second tissue filler materials can include any one or more of a collagen, an elastic fiber, fibronectin, a glycosaminoglycan, a proteoglycan, calcium hydroxyapatite, solar elastosis, elastin, fibrillin, fibulin, decorin, biglycan, hyaluronic acid, versican, silicone, poly L-lactic acid, and a polymethyl methacrylate microsphere.

In another aspect, the invention features tissue filler compositions that include one or more biologically active factors and a matrix material, wherein the biologically active factor is encapsulated or entrapped by, or complexed, aggregated, or otherwise incorporated with the matrix material, and wherein the biologically active factor stimulates production of an endogenous tissue filler material when implanted into a living tissue.

In these compositions, the matrix materials can further include a discrete absorption component as described herein. The matrix materials can include one or more of a polymethyl methacrylate, polyethylene vinyl-acetate, polytetrafluoroethylene, polyvinylchloride, polyamide, polyethylene, polysulfone, polyethersulphone, polypropylene, polydimethylsiloxane, polystyrene, polyphenylene oxide, cellulose, polypropylene oxide, polyvinylidene fluoride, and a polybutylenepolymethyl methacrylate. The biologically active factors can be or include any one or more of an epidermal growth factor, heparin, transforming growth factor-beta, transforming growth factor-alpha, platelet-derived growth factor, basic fibroblast growth factor, connective tissue activating peptides, beta-thromboglobulin, insulin-like growth factor, interleukins, nerve growth factors, colony stimulating factors, tumor necrosis factors, osteogenic factors, supernatant from tissue culture, and bone morphogenic proteins, and any combinations thereof.

In certain embodiments, these tissue filler compositions can further include a tissue filler material mixed with or associate with the matrix material. For example, tissue filler materials such as a collagen, an elastic fiber, fibronectin, a glycosaminoglycan, a proteoglycan, calcium hydroxyapatite, solar elastosis, elastin, fibrillin, fibulin, decorin, biglycan, hyaluronic acid, versican, silicone, poly L-lactic acid, and a polymethyl methacrylate microsphere can be used.

In another aspect, the invention features methods of implanting a tissue filler composition into a tissue by obtaining one of the tissue filler compositions described herein, and implanting an effective amount of the tissue filler composition into a tissue, e.g., into a desired location. In certain embodiments, the tissue can be skin, and the tissue filler composition can be used to treat a wrinkle, photoaging, an acne scar, a scar, or lipoatrophy.

In yet another aspect, the invention features methods of modifying, e.g., reducing the size of, a tissue implant that includes one or more of the tissue filler compositions described herein. These methods include locating a portion of the tissue implant that is to be reduced in size; and disrupting the matrix material or matrix-associated tissue filler material in the tissue implant to render the matrix material and the tissue filler material dispersible in the tissue, thereby causing the tissue implant to be modified, e.g., reduced in size.

In these methods, the matrix materials can include an absorption component, in which case the matrix materials or the matrix-associated tissue filler materials are disrupted by applying irradiation at a wavelength selected to be absorbed by the absorption component in an amount sufficient to disrupt at least a portion of the matrix-associated tissue filler material. In these methods, the disruption can also be used to release a biologically active factor from the matrix or microparticle, to be dispersed in the tissue.

The invention also features matrix-associated tissue filler materials that include a matrix material, and a tissue filler material, wherein the matrix-associated tissue filler is indispersible when implanted into living tissue, but wherein the matrix material, the tissue filler material, or both become dispersible when the matrix material is disrupted or when the tissue filler material is separated from the matrix material. These matrix-associated tissue filler materials can be implanted into a tissue as described herein, e.g., to treat a wrinkle, photoaging, an acne scar, a scar, or lipoatrophy. In these methods, the matrix material can be polymethyl methacrylate and the tissue filler can include a chondroitin sulfate, a proteoglycan, an elastin, or any combination thereof.

In accordance with the present invention, the tissue filler compositions can be applied to tissues (whether human or animal) including, but not limited to, skin, teeth, gums, dental ligaments tissue inside the mouth including the tongue (or other structures in the mouth), esophageal sphincter, iris, sclera, muscles, tendons, organs (e.g., urinary bladed or urinary bladder sphincter), brain, small and large intestines, uterus, tumors, and other cellular masses, or tissues from which tumors or masses have been removed, tissue beneath fingernails or toenails, or tissue lining internal body passages.

As used herein, a “microparticle” is a particle of a relatively small size, not necessarily in the micron size range, but generally covering particles of a size that can be implanted into a tissue and have an overall largest cross-sectional dimension of about 50 nm to about 100 microns or greater. In contrast, a “nanoparticle” is specifically a particle in the nanometer size range, for example, about 15 nm to about 500 nm. A micro- or nanoparticle may be of composite construction and need not necessarily be a pure substance, but can be made of a combination of materials. Micro- and nanoparticles can be spherical or any other shape. Both the micro- and nanoparticles can have a core and coating (or shell) made of a matrix material that surrounds the core. Alternatively, a micro- or nanoparticle can also be made of a matrix that envelops one or more smaller nanoparticles, without a separate or distinct coating and core.

As used herein, a “dispersible” substance (such as certain tissue filler materials that can be associated with a microparticle to provide a long-term tissue implant) is (1) dissolved by (and is soluble in) bodily fluids, for example, those within a cell or tissue, either in a short time (e.g., within a matter of a few minutes or hours) or over a period of days, weeks, or months; (2) metabolized (including digested) by living tissue and/or cells into one or more new products; and/or (3) of a size (on average no larger than about 50 nanometers, but in some cases necessarily much smaller, for example, less than about 5 nm), made of a material, and configured such that normal bodily processes result in its physical relocation from tissue (from cells or from extracellular matrix).

As used herein, an “indispersible” substance (such as a matrix, e.g., a coating, material, or an individual microparticle, or certain tissue fillers that need not be associated with a microparticle, or that can be aggregated with a matrix material without forming discrete microparticles) does not disintegrate, dissolve, or become metabolized in tissue. “Indispersible” microparticles are also large enough on average (generally greater than about 50 nm, but depending on the material as small as 5 nm or even smaller) and have a configuration on average such that when a plurality is implanted into tissue a sufficient number is retained to form a physical change in the tissue, even though some number of the individual microparticles may be relocated from the tissue implantation site through biological processes (such as lymphatic transport).

An “inert” or “biologically inert” substance (such as the matrix material that forms the coating of a microparticle or a tissue filler material itself), generally creates no significant biochemical, allergic, or immune response after the normal healing period when implanted into living tissue. A disruption of an otherwise “inert” microparticle may cause an inflammatory and/or immune response, when the tissue filler material within the microparticle leeches out, or when an agent designed to cause an inflammatory response leeches out of the microparticle.

A “microparticle-associated tissue filler material” is a tissue filler material that itself comprises a microparticle, that is encapsulated by, incorporated in, or entrapped with a matrix material to form a microparticle, or is complexed with, e.g., covalently or non-covalently bound with, a microparticle. The term “matrix-associated tissue filler material” is a broad term that includes microparticle-associated tissue filler materials as well as tissue filler materials that are aggregated or otherwise made indispersible and/or non-biodegradable in the presence of specific matrix materials, even without the formation of individual microparticles.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A is a schematic cross-sectional view of a microparticle-associated tissue filler material.

FIG. 1B is a schematic cross-sectional view of a vessel containing a liquid or gel tissue filler composition including microparticle-associated tissue filler materials and tissue filler material that is mixed with, but not associated with any microparticles.

FIG. 2 is a schematic cross-sectional view of a microparticle containing tissue filler material nanoparticles.

FIG. 3 is a schematic cross-sectional view of a microparticle containing sub-microparticles comprising encapsulated tissue filler materials.

FIG. 4 is a schematic cross-sectional view of a microparticle containing a first tissue filler material encapsulated within a core, and a sub-microparticle comprising a different tissue filler material encapsulated within.

FIG. 5 is a schematic cross-sectional view of another embodiment of a microparticle that does not have a distinct shell and core configuration.

### DETAILED DESCRIPTION

Described herein are tissue filler compositions including at least one tissue filler material that is encapsulated or entrapped by, or is complexed, aggregated, or otherwise incorporated with, a biologically inert, and non-biodegradable vehicle or matrix, to form an indispersible and non-biodegradable composition. In some embodiments, the matrix or the tissue filler itself forms indispersible, non-biodegradable microparticles. In other embodiments, the tissue filler material may itself be dispersible, but is made indispersible by its association with a microparticle, e.g., to form a gel. In some embodiments, the tissue filler microparticles include a discrete absorption component or other means to facilitate removal of the microparticles from the tissue, if desired, after implantation. In some embodiments, the tissue filler materials within these microparticles can be a gas, such as air, or a liquid such as saline or purified water, that are easily dispersed once the matrix is disrupted.

In other embodiments, the tissue filler composition includes a combination of one or more tissue fillers that are not associated with a matrix or microparticle, and the matrix- or microparticle-associated tissue filler materials described herein. This type of arrangement may be beneficial, for example, to provide a composition that includes a portion of a filler material that cannot be altered, but that may have certain desirable characteristics such as space filling or flow characteristics, and another portion of the tissue filler composition that can be altered, e.g., to reduce an overly aggressive implantation or to reduce the size of an implant after the body has caused a reaction that induces an increase in the volume of the tissue implantation site due to the localized production of materials by the body.

In another embodiment, the combination of the matrix and the tissue filler material may also include a discrete absorption component as part of a matrix forming a microparticle, or as part of an indispersible composition that does not include individual microparticles. This configuration is particularly useful to correct a filling of a tissue that was not performed as desired. Specifically, targeting the discrete absorption component with exogenous energy can remove a part of or all of the tissue filler microparticles or matrix.

The ratio of the matrix- or microparticle-associated tissue filler material to the non-associated filler material may be chosen as desired. Preferably, the ratio is from about 1:3 to about 3:1, for example, 1:1, 1:2, or 2:1, by weight.

In yet another embodiment, the tissue filler material itself serves as a “matrix” that can be is used to form a microparticle that encapsulates or entraps, or is otherwise complexed, aggregated, or associated to become indispersible. Such tissue filler materials can include a discrete absorption component. In this case, the discrete absorption component disrupts the tissue filler material by heating and melting or vaporizing the tissue filler material itself, aiding in its removal or alteration. This tissue filler material can be used alone or in any combination with the tissue fillers described herein.

Microparticles

Microparticles capable of providing selectively removable tissue implants must meet several criteria. First, the microparticles must be removed as desired by a specific externally applied treatment. Second, the microparticles must be indispersible, as described herein, in the tissue under normal physiological conditions. Third, as a safety precaution, any component of the microparticles which will at any time (such as during implantation or removal or while the implant resides in the tissue) come into contact with the tissue must be substantially biologically inert, unreactive, and/or safely metabolized. Described herein are microparticles that meet these three requirements.

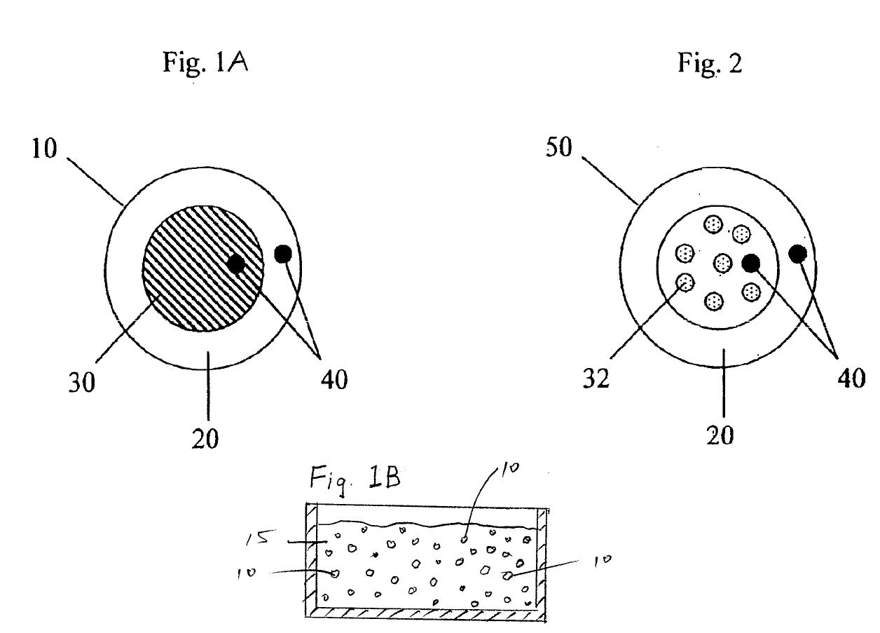

FIG. 1A shows a basic microparticle-associated tissue filler 10, which includes a coating 20 encapsulating a core containing tissue filler material(s) 30. As shown in microparticle 50 of FIG. 2, the core may contain discrete tissue filler nanoparticles 32 or could be filled with a liquid (e.g., sterilized water or saline) or gaseous (e.g., sterilized air, oxygen, or nitrogen) tissue filler material. FIG. 1B shows a cross-sectional view of a vessel containing a tissue filler composition that includes microparticle-associated tissue filler materials 10 dispersed or mixed within a tissue filler material 15, e.g., a semisolid, liquid, or gel, that is not associated with any microparticles, as described herein.

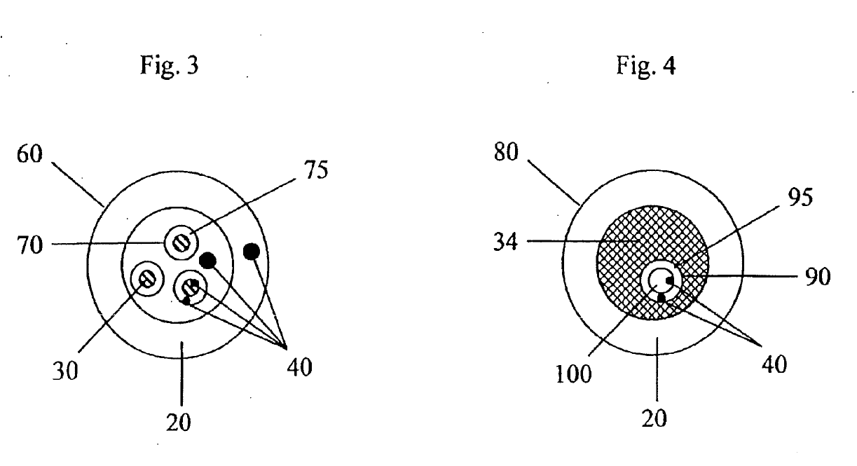

In certain cases, as depicted in FIG. 3, it may be useful to encapsulate a plurality of composite sub-microparticles 70, comprising tissue filler material(s) 30 and a substantially transparent coating 75 (which may or may not be the same material as used in coating 20), in coating 20 to form microparticle 60. Sub-microparticles 70 can be any size as long as they fit within the microparticle 60. The tissue filler materials can be colorless, transparent, or can include a color to mimic the surrounding tissue. In certain situations, it may be desirable to include a chromophore or pigment within the microparticles that either mimics the color of the tissue to be implanted or a color that has a desired cosmetic appearance, such as red, blue, or black. Cosmetic pigments of many different varieties and colors are known and can be used herein.

In another embodiment, illustrated schematically in FIG. 4, a tissue filler material 34 fills the core of the microparticle, and can optionally include one or more composite sub-microparticle(s) 90 (comprising different tissue filler(s) or colored pigments 100 and coating 95) are encapsulated in coating 20 to form microparticle 80.

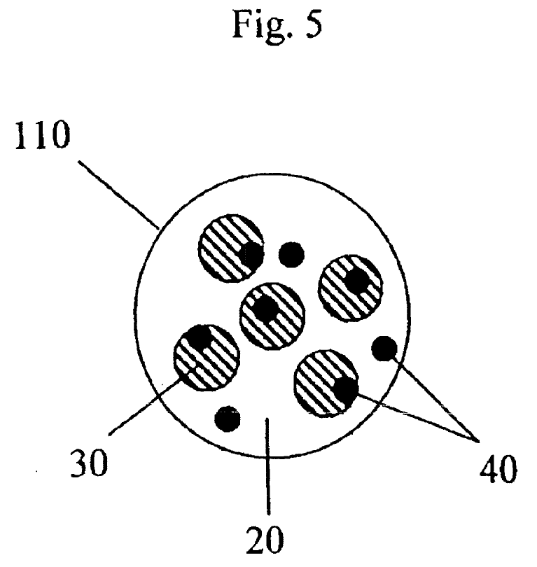

FIG. 5 depicts an optional configuration for the microparticle in FIG. 1, where two or more nanoparticles of tissue filler material(s) 30 can be present within the coating 20 of a single microparticle 110. Analogous multi-core versions of the microparticles in FIGS. 2 to 4 can also be constructed.

Generally, coating 20 and/or 75 or 95 is made from any matrix material described herein that is indispersible (at least once formed into a microparticle of a sufficient size (and is therefore generally retained in tissue) and is biologically inert under physiological conditions. The coating can have a thickness ranging from about 0.05 r (about 86% core loading, 14% coating, by volume) to about 0.6 r (about 6.4% core loading, 93.6% coating, by volume), where r is the microparticle radius. The coating can be from about 10 to about 95 percent of the total volume of a microparticle. The coating can be transparent, colorless, or can include desired pigments. Pigments can also be included in the core.

The microparticles also generally include one or more absorption component(s) 40 that are incorporated into coatings 20, 75, or 95, and/or mixed with tissue filler material(s) 30, tissue filler nanoparticles 32, or subparticle(s) 100 in the core or within a microparticle that does not include a recognizable core.

In general, the microparticles are constructed to contain dispersible tissue filler materials that are removed when microparticles are made permeable, for example, by rupture of a coating. Alternatively, for the microparticles that have no specific coating and core, the entire microparticle is disrupted and broken into sufficiently small particles to be removed by bodily functions.

More specifically, according to the one embodiment, microparticles 10, 50, 60, and 110 can contain dispersible tissue filler materials and/or chromophore(s) 30 or 32. Tissue implants made using tissue filler compositions including these microparticles can be removed when desired using a method wherein the tissue implant is exposed to specific electromagnetic radiation that ruptures the microparticles. For example, the microparticles can rupture as the result of heating, for example, when the coating 20 and/or 75, tissue filler material(s) 30 or 32, or additional absorption component(s) 40 absorb the specific radiation. In this embodiment, when the tissue filler materials are dispersed from the tissue implantation site, any undesired bulging or protrusion of filler material slowly disappears. This result can occur over the course of several minutes to several hours, days, or even weeks following irradiation.

Microparticles 10, 50, and 110, which contain dispersible tissue filler material(s) 30 and 32 can also be constructed with porous coatings such that the filler material(s) leaches out and is dispersed over time, to allow natural materials produced by the body, e.g., collagen, to slowly replace the leached filler material without increasing the size of the implant. If desired, these microparticles can also be designed in advance for removal using specific electromagnetic radiation as in the above description.

Certain aspects of the design of the several microparticles described herein may be interchanged or omitted, yielding useful microparticles. These and other types of microparticles are within the scope of the invention and will be useful if they are in the size range capable of providing indispersible tissue implants and if they satisfy the three criteria outlined above. The microparticles can be made using known methods, e.g., as described in Anderson et al., U.S. Pat. No. 6,814,760, and Mathiowitz et al., PCT/US2007/0688, which are each incorporated herein in their entirety.

Tissue Fillers

In some embodiments, the tissue filler materials include extracellular constituents of the tissue into which it is desirable to implant the filler. In the case of the dermis, these materials include collagens (all types), elastic fibers, fibronectin, glycosaminoglycans, and proteoglycans. The types of extracellular constituents may be found in Principals and Techniques of Cutaneous Surgery, Chapter I, pages 1-22 (McGraw Hill), which is incorporated by reference herein in its entirety. The filler materials may also include, but are not limited to, elastin, fibrillin, fibulin, decorin, biglycan, hyaluronic acid, calcium hydroxyapatite, silicone, cells, and poly L-lactic acid. Commercially available fillers include Restylane®, and Artecoll®, which is composed of PMMA microspheres (30 to 42 microns in size) suspended in a water-based carrier gel (composed of 3.5% bovine collagen and 96.5% buffered, isotonic water).

Chondroitin sulfate (CS) proteoglycan can also be used as a filler material, either alone or co-distributed with other materials, such as elastin or collagen. As used herein, the CS proteoglycans include versican, decorin, or combinations thereof.

Also, the fillers may be those based on solar elastosis and/or other fillers based on or derived from elastic fibers of humans and animals, as described in Bernstein, U.S. Published Patent Application No. US 2007/0071729 A1, which is incorporated herein by reference in its entirety. Specifically, the filler can be a mammalian elastic material derived from a site of solar elastosis or from at least one sun-damaged site in a mammal. In some embodiments, the mammal is a human; for example, the elastic material can be derived from the same human into which it is later implanted. Alternatively, the elastic material can be derived from a cadaver; created in vitro, e.g., using cells derived from Chinese hamster ovary cells; purified from the milk or tissues of a transgenic animal (e.g., a goat, sheep, cow, or mouse expressing a human gene or genes for one or more constituents of the elastic material); or a combination thereof.

Chronic ultraviolet radiation results in the accumulation of abnormal elastic fibers in the skin, termed “solar elastosis,” because they stain similar to elastin. These abnormal elastic fibers account for the majority of changes associated with an aged appearance in chronically sun-damaged skin, including fine lines and wrinkles and sagging skin. Dramatic alterations of the superficial dermis are responsible for the deep wrinkles and laxity that occur in photodamaged skin. Immunohistochemical staining demonstrates that these poorly formed clumps of elastic fibers comprising solar elastosis are comprised of the normal constituents of elastic fibers, e.g., elastin, fibrillin, fibulin, versican, and glycosaminoglycans (GAGs).

One of the interesting features of solar elastosis is that it is quite resistant to degradation, and persists throughout the lifetime of an individual. Most treatments directed at rejuvenating photodamaged skin cause an accumulation of new skin, including collagen and the previously mentioned normal constituents of elastic fibers, e.g., elastin and GAGs, in the very superficial dermis. Beneath this zone of normal-appearing skin, there still resides a significant amount of solar elastotic material despite even aggressive attempts at rejuvenating photodamaged skin. For example, even after carbon dioxide laser resurfacing, which removes not only the epidermis, but also a portion of the superficial dermis, there is still significant solar elastosis left in the dermis. This persistence of solar elastosis may be one of the biggest barriers to rejuvenating chronically sun-damaged skin. However, this persistence of solar elastotic material may also be seen as a potential benefit when used to prepare tissue filler materials for humans and other mammals.

Photoaged skin is currently treated with topical applications of anti-aging compounds, such as tretinoin or glycolic acid, dermabrasion, or laser resurfacing, the latter two of which remove the surface of the skin causing massive inflammation. All of these treatments fail to remove 100% of the solar elastotic material. Solar elastotic tissue is significantly resistant to removal by an inflammatory response. This resistance to removal is one of the properties that makes solar elastotic tissue an ideal filler material for the skin and other organ systems such as the esophageal and urinary tract sphincters, deficits in connective tissue, muscle or bone and other organs. In addition, various components of the elastic fibers are also variably resistant to digestion by proteases and other inflammatory mediators such as Interferon-gamma (IFN-γ), transforming growth factor-alpha (TGF-α), and Interleukin-1 (IL-1). Thus, the solar elastotic materials are ideal filler agents.

When fully encapsulated, the tissue filler material can include various sterilized gases, e.g., nitrogen, oxygen, carbon dioxide, or air, as well as various liquids such as sterilized saline or water.

Matrix Materials

The matrix can be made from or can include various types of materials. For example, the matrix can be made from or include a material that is non-biodegradable, e.g., a material that is designed to remain in tissue indefinitely, or a material that is biodegradable or bioabsorbable. The biodegradable matrix materials are used either in situations when a slow removal of the tissue filler material from a tissue implant is desirable, or when they are coated with a non-biodegradable matrix that will render the combined composition indispersible and non-biodegradable.

Examples of biodegradable or bioabsorbable materials include, but are not limited to, alginates, such as zinc alginates, poly(vinyl alcohols), polyanhydrides, polylactides, poly(glycolic acid), poly-L-lactic acids, poly D-lactic acids, poly-lactic-glycolic acids, and triblock copolymers of poly caprolactone-polyethylene glycol-poly caprolactone. These and other examples of various types of matrices that can be used in the present methods and compositions can be found, for example, in U.S. Pat. Nos. 6,814,760; 6,013,122; 3,981,303; 3,986,510, and 3,995,635, as well as in International Application No. PCT/US2006/030037 and Provisional Application No. 60/744,224, each of which is incorporated herein by reference in its entirety.

In some embodiments, the matrix material is non-biodegradable. Examples of such materials include polymethyl methacrylate (PMMA), which can be used, e.g., in the form of microcapsules, microparticles, or beads that include, e.g., encapsulate, the filler material. Other examples of non-biodegradable polymers that are believed to be biocompatible include polyethylene vinyl-acetate, polycarbonates, polytetrafluoroethylene (PTFE), polyvinylchloride (PVC), polyamide (PA, nylons), polyethylene (PE), polysulfones, polyethersulphone, polypropylene (PP), polydimethylsiloxane (PDMS, or silicone rubber), polystyrene, polyphenylene oxide, cellulose and its derivatives, polypropylene oxide (PPO), polyvinylidene fluoride (PVDF), and polybutylene. Other plastics and organic polymers are also useful as matrix materials, such as parylenes, polyvinyl acetates, urea formaldehyde, melamine formaldehyde, ethylene acrylate, cyanoacrylates, butadiene styrene, and specifically biocompatible materials such as EPO TEK® 301 and 301-2, manufactured by Epoxy Technology, Billerica, Mass.

Discrete Absorption Components

In some embodiments, the tissue filler material itself, or in combination with a matrix, also includes at least one discrete absorption particle or component, which is designed to absorb a specific, exogenous energy. These absorption particles are shown as component 40 in FIGS. 1A, 2, 3, 4, and 5. When implanted into the tissue, the filler will generally be either internalized by cells or remain in the extracellular matrix. To remove or alter the tissue filler, exogenous energy is applied to the tissue. Once a sufficient amount of such exogenous energy has been applied, the discrete absorption particles can rupture, or otherwise heat and disrupt, the matrix (or the tissue filler material), releasing the now dispersible filler material, which can dissipate, dissolve, or be removed by natural biological processes, such as via the lymphatic system or through expulsion. In some embodiments, the particle is embedded in a coating of the matrix and acts like an “egg tooth” that breaks open the coating, e.g., of a microparticle, to allow the filler material to be released from the material coating. In some embodiments, the matrix, discrete absorption particle, and filler material form a substantially homogeneous mixture, with the particles distributed throughout a composition that includes both the matrix and filler material.

Anderson et al., U.S. Pat. No. 6,814,760 and Provisional Application No. 60/744,224 (PCT WO 2007/115291), each of which is incorporated herein by reference in its entirety, both describe various materials for use as discrete absorption components.

Non-limiting examples of materials that can be used to form discrete absorption particles include metals, such as gold, silver, and platinum. Particles formed from such metals, particularly those on a nanometer scale and their derived nanostructures, exhibit specific absorption features associated with the plasmon resonance of conduction electrons confined in the nanoparticle. The absorption frequency, such as absorption maxima or color, and other absorption features, such as the shape and the width of the absorption peaks, of these nanoparticles and other metallic nanostructures, such as nanoshells, are determined by the type of material, size and shape of nanoparticles and nanostructures, size distribution, and the environment that surrounds these particles (Hutter and Fendler, Advanced Materials, 2004, 16(19), p. 1685). For example, gold nanoparticles have a strong plasmon resonance absorption at a wavelength of 520 nm. Silver nanoparticles have a plasmon resonance absorption at 390 nm. The absorption cross-section of metallic nanoparticles and nanostructures, such as nanoshells, is substantially larger than that of filler materials and matrix materials, which is sufficient to efficiently rupture or disrupt the matrix and/or tissue filler microparticles. It should be noted that the sizes of discrete absorption particles may vary depending on the desired filler material and is not necessarily on the nanometer scale.

Metallic and composite metallic discrete absorption nanoparticles and nanostructures that exhibit plasmon resonance can be prepared in various shapes, such as nanospheres, nanoshells, rods, rings, disks, and cubes. Several different preparation techniques, such as colloidal metallic preparation methods, microemulsions, surfactant stabilized micelles, reverse micelles, surfactant vesicles, as well as laser ablation methods, vacuum deposition, and electron beam lithography can be used to form these structures (Advanced Materials, 2004, 16(19), p. 1685 and its references).

Useful materials include metallic nanoshells (Halas et al. Journal of Optical Society of America B, 1999, 16(10), p 1824; Halas et al. Science, 2003, 302, p. 419). Nanoshells are optically tunable nanoparticles composed of a dielectric (for example, silica) core coated with an ultra-thin metallic layer. Gold nanoshells, for example, those developed by Nanospectra Biosciences, Inc., Houston, Tex., have physical properties, particularly a strong plasmon resonance, similar to a gold colloid. The maximum absorption (plasmon resonance) of nanoshells can be varied over a wide range by varying the ratio of inner to outer diameter of the shell, yielding plasmon resonance tunable from about 600 nm to greater than about 1000 nm (Halas, Nano Letters, 2003, 3(10), p. 1411). For example, gold silica nanoshells with a 100 nm core and a 5 nm shell thickness show a maximum absorption at about 1000 nm, while those having the shell thickness of 20 nm have a maximum absorption at about 700 nm.

Single and multiple layer nanoshells ranging in size from a few nanometers to a few hundred nanometers may be prepared and used as discrete absorption particles in tissue filler microparticles. Such nanoshells may be, for example, about 200 to about 800 nm in diameter (Halas et al. Science, 2003, 302, p. 419). Again, the sizes will depend on the desired filler material.

The metallic nanoparticles and nanostructures can be stabilized by covalently bound thiol and disulfide functionalized monolayers. If desired, the surface of metallic nanoparticles can be functionalized to induce a specifically desired immune response or to target specific surface receptors of antigen-presenting tissue cells to induce surface receptor mediated endocytosis, as discussed in Provisional Application No. 60/587,864 (International Application No. PCT WO 2007/115291), which are incorporated herein by reference in their entirety.

Other materials that can be used as discrete absorption particles with IR and near IR absorption include those disclosed in U.S. Pat. No. 6,800,122 to Anderson. In particular, these materials include, but are not limited to, graphite and other forms of carbon and metal oxides, such as iron oxide (red/brown or black), glasses (BG-7 and KG-3 filter glass made by Schott, Inc.), cyanine dyes (including indocyanine green and other colors), phthalocyanine dyes (green-blue), and pyrylium dyes (multiple colors).

Materials with magnetic properties can also be used to form discrete absorption particles. Specifically, black iron oxide particles, such as superparamagnetic iron oxide (SPIO, particle size greater than about 50 nm), ultrasmall superparamagnetic iron oxide (USPIO, particle size smaller than about 50 nm), and monodisperse iron oxide nanoparticles (MION, particle size smaller than about 20 nm) can be used. Superparamagnetic iron oxide consists of nonstoichiometric microcrystalline magnetite cores, which are coated with dextrans (in ferumoxides) or siloxanes (in ferumoxsils). The most common form of iron oxide that may be used is magnetite, which is a mixture of Fe₂O₃ and FeO. Fe₃O₄ may be used in lieu of FeO. These materials are available as tissue specific MRI contrast agents (e.g., Feridex®, Endorem™, GastroMARK®, Lumirem®, Sinerem®, and Resovist®. USPIO particles are also available as MRI contrast agents (e.g., Sinerem®, Combidex®, and Clariscan™).

If desired, magnetic iron oxide nanoparticles can be functionalized to induce a specifically desired immune response or to target specific surface receptors of antigen presenting cells in the tissue to induce surface receptor mediated endocytosis, as disclosed in Provisional Application No. 60/587,864 (International Application No. PCT WO 2006/019823), which are incorporated herein by reference in their entirety.

The discrete absorption components can be used with various types of matrices, or with tissue fillers alone if they are in an indispersible and/or non-biodegradable form, to prepare removable or alterable microparticles as described herein. These matrices may be bioerodable or biodegradable. The discrete absorption component may be used for a better control of how the tissue is filled. For example, if the application of the filler results in an undesired bump or protrusion, the discrete absorption component can be used to remove or alter at least the microparticles of the filler deposit on demand without waiting for the microparticles to dissipate or be dissolved.

Biologically Active Factors

In yet another embodiment, in lieu of or in addition to, a combination of the filler material and the microparticles, the microparticles may encapsulate or entrap, or may be otherwise complexed, aggregated, or associated with a material that stimulates tissue generation or other agents. Non-limiting examples of biologically active factors that may be used include epidermal growth factor, heparin, transforming growth factor-beta, transforming growth factor-alpha, platelet-derived growth factor, basic fibroblast growth factor, connective tissue activating peptides, beta-thromboglobulin, insulin-like growth factor, interleukins, nerve growth factors, colony stimulating factors, tumor necrosis factors, osteogenic factors, supernatant from tissue culture, bone morphogenic proteins, and combinations thereof. For example, if the tissue is dermis, these biologically active factors may include transforming growth factor-β (TGF-β) and/or platelet-derived growth factor (PDGF), discussed in Burnstein et al., “Transforming Growth factor-β Improves Healing of Radiation Impaired Wounds,” J. Invest. Dermatol. 97:430-434, 1991, which is incorporated herein by reference in its entirety. Optionally, as in the above embodiments, a discrete absorption component can also be used.

When the microparticles or matrix materials release the biologically active factors, they are able to prompt the organism to generate the needed filler materials in lieu of supplying such materials externally. In this case, there is a lesser likelihood of an adverse reaction by the body, because the generated material is not viewed as foreign.

Also, the size and amount of the matrix-associated filler materials can be reduced. To place the matrix with the filler materials into the extracellular matrix of a tissue, the matrix/filler material microparticles (with or without the discrete absorption component), are preferably about 15-50 microns, e.g., 32-40 microns in size. However, biologically active factors as described herein can be placed inside tissue cells as described in International Application No. PCT/US06/30037, which is incorporated herein in its entirety, and may be on a nanometer scale.

Methods of Application and Use

The new compositions described herein can be used to remodel a tissue that has been damaged. Such damage may have been caused, e.g., by normal aging, exposure to the sun, or by trauma. The matrix-associated tissue filler materials and tissue filler compositions described herein can be applied by conventional methods known in the art. It is understood that the tissue filler materials can be combined in a suitable carrier, which may include various active agents and/or various types of fillers that achieve certain effects. For example, the carrier may include one or more biologically active factors (in addition to those discussed above) to aid in the healing, regrowth, stability, and/or longevity of the natural tissue or to aid in the stability or longevity of the tissue filler material. The carriers can also be sterilized water, oils, or saline.

Tissue filler materials can be used for a myriad of applications in mammals. One of the widest uses recently has been for cosmetic augmentation of skin for cosmetic or functional purposes. Recently a number of tissue fillers have been approved for treating fine lines and wrinkles that result from aging, e.g., photoaging. Fillers are injected via a syringe intra-dermally to puff-up the skin resulting in diminution of the appearance of the wrinkle, fine line, or skin fold such as the nasolabial fold; or they may be inserted surgically through an incision in the skin. In addition, fillers have been used for augmenting loss of the fatty layer of skin as occurs, e.g., in HIV infected people.

For example, the compositions described herein can be used to augment the skin or subcutaneous fat of a human. This method is particularly useful for treating common skin conditions, such as wrinkles, photoaging, acne scarring, scars, or HIV-associated lipoatrophy. This method generally comprises implanting or injecting into the skin or subcutaneous fat of the human, elastic material described above, including solar elastosis and/or the described fillers. This same method can be used to prevent or reduce scarring of the skin of a human from various conditions.

In addition to cosmetic uses, fillers have been used to augment many tissues that have functional alterations that impair normal function or cause pain. For example, intervertebral discs can rupture or herniate and cause pain, resulting in numerous days of lost productivity from an individual and loss of quality of life due to pain. Filler materials may help in maintaining the proper intervertebral disc dimensions or prevent adhesions during surgery.

A material that is not seen as foreign by the immune system or that is not easily digested away would be preferred. Using such a filler to protect organ systems during any type of surgery from adhesions would be quite useful at preventing or reducing post-operative complications. Thus, agents that can prevent adhesions can also prevent scarring. The fact that solar elastotic tissues are resistant to degradation may also confer the ability to prevent tissue reaction and thus prevent scarring and adhesions. These complications are common after my types of surgery including, but not limited to, abdominal procedures, orthopedic procedures and the like.

Tissue fillers have also been used to augment the urethral sphincter in cases of urethral sphincter insufficiency. In addition, fillers have been used to augment the esophageal sphincter to help prevent acid reflux disease, which in turn can result in changes to the esophagus including Barratt's esophagus and possibly esophageal cancer. Fillers are often used in dental practice to augment and support the teeth, bones, and soft tissues of the mouth.

In one aspect of the present disclosure, there is provided a method of augmenting tissue or bone in or around the mouth by implanting or injecting the tissue filler materials described herein into the bone or tissue in or around the mouth. As used herein, “in or around the mouth” is intended to encompass tissues and bones in the jaw and/or mouth, such as the soft tissue in the mouth. This method can be used, e.g., to treat mammals suffering from gum disease, such as gingivitis, or to augment the teeth, dental ligaments, or other structures in the mouth.

In addition, the new tissue fillers can be used to augment bone. In some embodiments, the filler not only takes up space, but can also stimulate repair, serving as a provisional matrix for the ingrowth of the surrounding tissue such as skin or bone.

The methods described herein can also be used to augment other tissues in the body. Non-limiting examples of other tissues that can be augmented include the urinary bladder or the urinary bladder sphincter, and the esophageal sphincter.

If the tissue filler compositions are implanted incorrectly (e.g., in the wrong location or in too large an amount), or if they induce the body to produce endogenous tissue fillers that cause the implant to enlarge undesirably, they can be treated to remove the implant, to reduce the overall size of the implant, or to otherwise modify the implant. In particular, the microparticles in the composition, or the matrix-associated tissue filler materials in the composition, can be treated to apply energy that causes the microparticles or matrix to disrupt.

For example, the microparticles containing an absorption component can be treated with electromagnetic radiation (e.g., laser energy) in the visible (400-750 nm) to near-infrared (near-IR; 750-1100 nm) regions of the spectrum, to induce a structural or morphological change. The absorption of energy by the microparticles will break them down, releasing thermal, kinetic, and mechanical energy into the tissue, e.g., the dermis. This process facilitates removal of the microparticles from the tissue by (1) reducing the overall particle size or rupturing of any coating that is part of the microparticles; and (2) promoting the infiltration of leukocytes (such as macrophages) to the region containing the microparticles or matrix.

### EXAMPLES

The invention is further described in the following examples, which do not limit the scope of the invention described in the claims.

### Example 1

Approximately 500 mg of polyethylene vinyl acetate microparticles that are between 2 and 50 microns in diameter and contain between 0.1% (w/w) and 10% (w/w) colloidal gold are suspended in 1 ml of distilled water. Upon injection into a tissue site, these microparticles occupy space within the tissue and change the shape, texture, or morphology of the tissue.

Some or all of these microparticles can later be removed, if required, by treatment with laser energy in the visible (400-750 nm) to near-infrared (near-IR; 750-1100 nm) regions of the spectrum to induce a structural change and break down the microparticles. This facilitates removal of the microparticles from the dermis by (1) reducing the overall size of the microparticles and breaking any coatings of the microparticles; and (2) promoting the infiltration of leukocytes (such as macrophages) to the region containing the beads.

### Example 2

Approximately 500 mg of polymethylmethacrylate microparticles that are between 2 and 50 microns in diameter and contain between 0.1% (w/w) and 10% (w/w) colloidal gold are suspended in 1 ml of distilled water. Upon being injected into a tissue site, these microparticles can occupy space within the tissue and change the shape, texture, or morphology of the tissue. These microparticles can later be removed, as required, as described in Example 1.

### Example 3

Approximately 1 ml of a delivery vehicle comprised of a 50% (v/v) concentration of chondroitin sulfate (CS) proteoglycan:elastin (between 1:3 and 3:1) that carries approximately 500 mg of polymethylmethacrylate (PMMA) microparticles of Example 2 is delivered to a tissue site. As in Examples 1 and 2, this slurry occupies space within the tissue and change the shape, texture, or morphology of the tissue. The PMMA can later be removed, if desired, as described in Example 1.

### Example 4

Approximately 500 mg of polyethylene vinyl acetate microparticles that are between 2 and 50 microns in diameter and contain between 0.1% (w/w) and 10% (w/w) iron oxide are suspended in 1 ml of distilled water. Upon injection into a tissue site, these microparticles occupy space within the tissue and change the shape, texture, or morphology of the tissue. Some or all of these microparticles can later be removed, if required, as described in Example 1.

### Example 5

Approximately 500 mg of polymethylmethacrylate microparticles that are between 2 and 50 microns in diameter and contain between 0.1% (w/w) and 10% (w/w) iron oxide are suspended in 1 ml of distilled water. Upon injection into a tissue site, these microparticles can occupy space within the tissue and change the shape, texture, or morphology of the tissue. They can later be removed as described in Example 1.

### Example 6

Approximately 500 mg of polymethylmethacrylate (PMMA) microparticles that are between 2 and 50 microns in diameter and contain between 0.1% (w/w) and 10% (w/w) iron oxide and an effective amount of insulin-like growth factor, are suspended in 1 ml of distilled water. Upon injection into a tissue site of a mouse, these microparticles can occupy space within the tissue and change the shape, texture, or morphology of the tissue. Thereafter, a portion of the PMMA microparticles forming the tissue implant in the mouse are disrupted as described in Example 1 to reduce the size of the implant and to release the insulin-like growth factor. Upon disruption, 2.0 mm biopsies are taken in the vicinity of the tissue implant and observed under hematoxylin-eosin staining at 0, 2, 4, 6, 12, 24, 48, 72 hours after disruption, as well as at one week after disruption. The results indicate an inflammatory response.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A tissue filler composition comprising
a first tissue filler material, and
a matrix-associated tissue filler material comprising a matrix material and a second tissue filler material, wherein the matrix-associated tissue filler is indispersible when implanted into living tissue, but becomes dispersible when the matrix material is disrupted or when the second tissue filler material is separated from the matrix material.

2. The tissue filler composition of claim 1, wherein the matrix-associated tissue filler material further comprises a discrete absorption component.

3. The tissue filler composition of claim 1, wherein the first and second tissue filler materials comprise the same material.

4. The tissue filler composition of claim 1, wherein the first and second tissue filler materials comprise different materials.

5. The tissue filler composition of claim 1, wherein the first tissue filler material and the matrix-associated tissue filler material are mixed in a ratio of about 3:1 to about 1:3.

6. The tissue filler composition of claim 1, wherein the first tissue filler material and the matrix-associated tissue filler material are mixed in a ratio of about 1:1.

7. The tissue filler composition of claim 1, wherein the matrix material comprises one or more of a polymethyl methacrylate, polyethylene vinyl-acetate, polytetrafluoroethylene, polyvinylchloride, polyamide, polyethylene, polysulfone, polyethersulphone, polypropylene, polydimethylsiloxane, polystyrene, polyphenylene oxide, cellulose, polypropylene oxide, polyvinylidene fluoride, and a polybutylenepolymethyl methacrylate.

8. The tissue filler composition of claim 1, wherein the matrix material comprises a polymethyl methacrylate.

9. The tissue filler composition of claim 1, wherein the matrix material comprises one or more of an alginate, poly(vinyl alcohol), polyanhydride, polylactide, poly(glycolic acid), poly-L-lactic acid, poly D-lactic acid, poly-lactic-glycolic acid, and a triblock copolymer of poly caprolactone-polyethylene glycol-poly caprolactone.

10. The tissue filler composition of claim 8, wherein the polymethyl methacrylate forms a coating of a microparticle.

11. The tissue filler composition of claim 1, wherein either one or both of the first and second tissue filler materials comprise any one or more of a collagen, an elastic fiber, fibronectin, a glycosaminoglycan, a proteoglycan, calcium hydroxyapatite, solar elastosis, elastin, fibrillin, fibulin, decorin, biglycan, hyaluronic acid, versican, silicone, poly L-lactic acid, and a polymethyl methacrylate microsphere.

12. A tissue filler composition comprising a biologically active factor and a matrix material, wherein the biologically active factor is encapsulated or entrapped by, or complexed, aggregated, or otherwise incorporated with the matrix material, and wherein the biologically active factor stimulates production of an endogenous tissue filler material when implanted into a living tissue.

13. The tissue filler composition of claim 12, wherein the matrix material further comprises a discrete absorption component.

14. The tissue filler composition of claim 12, wherein the matrix material comprises one or more of a polymethyl methacrylate, polyethylene vinyl-acetate, polytetrafluoroethylene, polyvinylchloride, polyamide, polyethylene, polysulfone, polyethersulphone, polypropylene, polydimethylsiloxane, polystyrene, polyphenylene oxide, cellulose, polypropylene oxide, polyvinylidene fluoride, and a polybutylenepolymethyl methacrylate.

15. The tissue filler composition of claim 12, wherein the matrix material comprises polymethyl methacrylate.

16. The tissue filler composition of claim 12, wherein the biologically active factor comprises any one or more of an epidermal growth factor, heparin, transforming growth factor-beta, transforming growth factor-alpha, platelet-derived growth factor, basic fibroblast growth factor, connective tissue activating peptides, beta-thromboglobulin, insulin-like growth factor, interleukins, nerve growth factors, colony stimulating factors, tumor necrosis factors, osteogenic factors, supernatant from tissue culture, and bone morphogenic proteins, and any combinations thereof.

17. The tissue filler composition of claim 12, further comprising a tissue filler material mixed with the matrix material.

18. The tissue filler composition of claim 17, wherein the tissue filler material is associated with the matrix material.

19. The tissue filler composition of claim 17, wherein the tissue filler material comprises any one or more of a collagen, an elastic fiber, fibronectin, a glycosaminoglycan, a proteoglycan, calcium hydroxyapatite, solar elastosis, elastin, fibrillin, fibulin, decorin, biglycan, hyaluronic acid, versican, silicone, poly L-lactic acid, and a polymethyl methacrylate microsphere.

20. A method of implanting a tissue filler composition into a tissue, the method comprising
obtaining a tissue filler composition of claim 1, and
implanting an effective amount of the tissue filler composition into a tissue.

21. The method of claim 20, wherein the tissue is skin, and the tissue filler composition is used to treat a wrinkle, photoaging, an acne scar, a scar, or lipoatrophy.

22. The method of claim 20, wherein the matrix material comprises a polymethyl methacrylate.

23. A method of implanting a tissue filler composition into a tissue, the method comprising
obtaining a tissue filler composition of claim 12, and
implanting an effective amount of the tissue filler composition into a tissue.

24. A method of reducing the size of a tissue implant comprising a tissue filler composition of claim 1, the method comprising
locating a portion of the tissue implant that is to be reduced in size; and
disrupting the matrix-associated tissue filler material in the tissue implant to render the matrix material and the second tissue filler material dispersible in the tissue, thereby causing the tissue implant to reduce in size.

25. The method of claim 24, wherein the tissue filler composition comprises an absorption component, and wherein disrupting the matrix-associated tissue filler material comprises applying irradiation at a wavelength selected to be absorbed by the absorption component in an amount sufficient to disrupt at least a portion of the matrix-associated tissue filler material.

26. A method of modifying a tissue implant comprising a tissue filler composition of claim 12, the method comprising
locating a portion of the tissue implant that is to be reduced in size; and
disrupting the matrix material in the tissue implant to render the matrix material, the biologically active factor, or both, dispersible in the tissue, thereby releasing the biologically active factor.

27. A matrix-associated tissue filler material comprising
a matrix material, and
a tissue filler material,
wherein the matrix-associated tissue filler is indispersible when implanted into living tissue, but wherein the matrix material, the tissue filler material, or both become dispersible when the matrix material is disrupted or when the tissue filler material is separated from the matrix material.

28. A method of implanting a matrix-associated tissue filler material into a tissue, the method comprising
obtaining a matrix-associated tissue filler material of claim 27, and
implanting an effective amount of the matrix-associated tissue filler material into a tissue.

29. The method of claim 28, wherein the tissue is skin, and the matrix-associated tissue filler material is used to treat a wrinkle, photoaging, an acne scar, a scar, or lipoatrophy.

30. The method of claim 28, wherein the matrix material is polymethyl methacrylate and the tissue filler comprises a chondroitin sulfate, a proteoglycan, versican, an elastin, or any combination thereof.
